SEQUENCE LISTING (5 sequences)

SEQ ID NO: 1 - DNA, 2886 bp, Homo sapiens
  CDS (433)..(1701)
tgcgctcgggggggcgcgggcagatcgctggcttggagaggactgtggcaggtgagaggacctgtgcgtcgttctctgcagacctggccgccccgggtgtcagagagaggtggcgagttcgtgtccgccgggaattgttggctgttggggaaactttcctgcgaggtcagtcaaggctttgggggctctgttttgaatgtggatcaccactcggagtttactaatgtttacaaggctgcgcagtagggaaacggaagagttgggtgggggcaaaaaaaaaaattgaccgctgtccccgaaagtactagacgcctctgccgggaaggcgcccctgcgcgttctatccgagacgtagcttcgcagcgaattttataggaacttcattagcatattatggaacgtcccgcctcagccccccagtagttggctgtgatgtccttcgtggaatgtccttatcattcccctgcggaacgattggtcgctgaggcggatgaaggcgggcctagcgcaataactggtatgggtctgtgtttccgctgtcttcttttttctttttcggggaggagcggggtggagggtggacgagttgatttgaacgtcttcgggtcgctcggcctccagccttggattggttcttctcgctgctggggcgggccgtgctcttccgccctgcggtgtggttggttctcctcctggcctccgccctccaaatcggcgattcccataggcggcggctctcggggtgcggggcgagtctcccgctggcctcctccccattggctggaggcctggcgggtgtcgccccggcccctctccccgctcagcccggccactttcgggcgcggatttatagcagtagcagtgatcccgggcctgtgggctcggggccggggctgcagttcggaccgcctcccgcgacccgcggggccggctcggagacagtttcaggccgcatctttgctgacccgagggtggggccgcgcgtggccgtggaaacagatcctgaaggagctagacgagtgctacgagcgcttcagtcgcgagacagacggggcgcagaagcggcggatgctgcactgtgtgcagcgcgcgctgatccgcagccaggagctgggcgacgagaagatccagatcgtgagccagatggtggagctggtggagaaccgcacgcggcaggtggacagccacgtggagctgttcgaggcgcagcaggagctgggcgacacagcgggcaacagcggcaaggctggcgcggacaggcccaaaggcgaggcggcagcgcaggctgacaagcccaacagcaagcgctcacggcggcagcgcaacaacgagaaccgtgagaacgcgtccagcaaccacgaccacgacgacggcgcctcgggcacacccaaggagaagaaggccaagacctccaagaagaagaagcgctccaaggccaaggcggagcgagaggcgtcccctgccgacctccccatcgaccccaacgaacccacgtactgtctgtgcaaccaggtctcctatggggagatgatcggctgcgacaacgacgagtgccccatcgagtggttccacttctcgtgcgtggggctcaatcataaacccaagggcaagtggtactgtcccaagtgccggggggagaacgagaagaccatggacaaagccctggagaaatccaaaaaagagagggcttacaacaggtagtttgtggacaggcgcctggtgtgaggaggacaaaataaaccgtgtatttattacattgctgcctttgttgaggtgcaaggagtgtaaaatgtatatttttaaagaatgttagaaaaggaaccattcctttcatagggatggcagtgattctgtttgccttttgttttcattggtacacgtgtaacaagaaagtggtctgtggatcagcattttagaaactacaaatataggtttgattcaacacttaagtctcagactgatttcttgcgggaggagggggactaaactcaacctaacacattaaatgtggaaggaaaatatttcatttagcttttttattttaatacaagtaatattattactttatgaacaattttttttaattggccatgtcgccaaaaatacagcctatagtaaatgtgtttcttgctgccatgatgtatatccatataacaattcagtaacaaaggtttaaagtttgaagattattttttaaaaaggtaaatggttaaattttacatgacagatattttatttattggcctgttccccaaatggccattttaaaatgcttgggtacacttctcttaagtggtctagtcaaggaacctcaagtcatgcttttgctatcaccaatcatagtgtacccatctttaatttatatcaggtgtataaatgtacatttccaaatgaacttgcacttgttatattataattggaagtgcagtcagcagatgctgttgtgaagctaatgtcacaattatgtgcaaaggtgtgcttcctgctgtatgtgagctgtaaaaatgttacgtgaagaaataaatgaaacttggccagtttgttcctctagtagtatatttaattttgacataagtaacttttaaaatttgtcttaaaaatttatacaccagcaatttagacaaagccttaagcaaattttgtattattgttctcacttattattaataatgaagtagaagttacttaattgccagcaaataaatacgtgtcaaaaaagaatctgtattcagaccctgggtcaggaaattactgcccacttgtcaagttcagcccaccatctgtttgaacattatatgaagtttaaattttagtgtccataaataaagtttcagcggaacacaaaaaaaaaaaaaaaaaaaaaaaaaaaaaaaaaaaaaaaaaaaa

SEQ ID NO: 2 - protein, 422 aa, Homo sapiens
MSFVECPYHSPAERLVAEADEGGPSAITGMGLCFRCLLFSFSGRSGVEGGRVDLNVFGSLGLQPWIGSSRCWGGPCSSALRCGWFSSWPPPSKSAIPIGGGSRGAGRVSRWPPPHWLEAWRVSPRPLSPLSPATFGRGFIAVAVIPGLWARGRGCSSDRLPRPAGPARRQFQAASLLTRGWGRAWPWKQILKELDECYERFSRETDGAQKRRMLHCVQRALIRSQELGDEKIQIVSQMVELVENRTRQVDSHVELFEAQQELGDTAGNSGKAGADRPKGEAAAQADKPNSKRSRRQRNNENRENASSNHDHDDGASGTPKEKKAKTSKKKKRSKAKAEREASPADLPIDPNEPTYCLCNQVSYGEMIGCDNDECPIEWFHFSCVGLNHKPKGKWYCPKCRGENEKTMDKALEKSKKERAYNR

SEQ ID NO: 3 - DNA, 2897 bp, Homo sapiens
  CDS (873)..(1712)
gcgagttgcggtagttgctgtgtaccatggtctcggaggtttctgtcccgcggcccgttaggtcctggtcgggttttcagcgaagcaggccgctcccctgcgtttcccagcgggcgtgctgtgccgcccaacaggctctgcctccaagtgccaaaaactcctagtaaagtttgcgcctcgcccgccgtccacaccccagcggccctgacgctgtcccctccgcgaccctcgcctctggaaaaagtgacaggcaaggccacgcccccgcgagggccggcctcgagcccgcagcccccagggcctgggacggtgaggggcgtgaatgcggcggggggcggggccgttgccgggggagggggccggggcgcatgcgcgctgcgcagcggggctgaatgtttcccaagtgtttgaaactggtatttgggttttccacgttggacaagtgcggctcggcggccagcggagcgcgccccttcccgctgcccgctccgctcctctcttctacccagcccagtgggcgagtgggcagcggcggccgcggcgctgggccctctcccgccggtgtgtgcgcgctcgtacgcgcggcccccggcgccagccccgccgcctgagagggggcctgcgccgccggccggggcgtgcgcccgggagccaccgccaccgcggcccgcgccctcaggcgctggggtccccgcggacccggaggcggcggacgggctcggcagatgtagccgccgggccgaagcaggagccggcgggggggcgccgggagagcgagggctttgcattttgcagtgctattttttgaggggggcggggggtggaggaagcggaaagccgcgccgagtcgccggggacctccggggtgaaccatgttgagtcctgccaacggggagcagctccacctggtgaactatgtggaggactacctggactccatcgagtccctgcctttcgacttgcagagaaatgtctcgctgatgcgggagatcgacgcgaaataccaagagatcctgaaggagctagacgagtgctacgagcgcttcagtcgcgagacagacggggcgcagaagcggcggatgctgcactgtgtgcagcgcgcgctgatccgcagccaggagctgggcgacgagaagatccagatcgtgagccagatggtggagctggtggagaaccgcacgcggcaggtggacagccacgtggagctgttcgaggcgcagcaggagctgggcgacacagcgggcaacagcggcaaggctggcgcggacaggcccaaaggcgaggcggcagcgcaggctgacaagcccaacagcaagcgctcacggcggcagcgcaacaacgagaaccgtgagaacgcgtccagcaaccacgaccacgacgacggcgcctcgggcacacccaaggagaagaaggccaagacctccaagaagaagaagcgctccaaggccaaggcggagcgagaggcgtcccctgccgacctccccatcgaccccaacgaacccacgtactgtctgtgcaaccaggtctcctatggggagatgatcggctgcgacaacgacgagtgccccatcgagtggttccacttctcgtgcgtggggctcaatcataaacccaagggcaagtggtactgtcccaagtgccggggggagaacgagaagaccatggacaaagccctggagaaatccaaaaaagagagggcttacaacaggtagtttgtggacaggcgcctggtgtgaggaggacaaaataaaccgtgtatttattacattgctgcctttgttgaggtgcaaggagtgtaaaatgtatatttttaaagaatgttagaaaaggaaccattcctttcatagggatggcagtgattctgtttgccttttgttttcattggtacacgtgtaacaagaaagtggtctgtggatcagcattttagaaactacaaatataggtttgattcaacacttaagtctcagactgatttcttgcgggaggagggggactaaactcaacctaacacattaaatgtggaaggaaaatatttcatttagcttttttattttaatacaagtaatattattactttatgaacaattttttttaattggccatgtcgccaaaaatacagcctatagtaaatgtgtttcttgctgccatgatgtatatccatataacaattcagtaacaaaggtttaaagtttgaagattattttttaaaaaggtaaatggttaaattttacatgacagatattttatttattggcctgttccccaaatggccattttaaaatgcttgggtacacttctcttaagtggtctagtcaaggaacctcaagtcatgcttttgctatcaccaatcatagtgtacccatctttaatttatatcaggtgtataaatgtacatttccaaatgaacttgcacttgttatattataattggaagtgcagtcagcagatgctgttgtgaagctaatgtcacaattatgtgcaaaggtgtgcttcctgctgtatgtgagctgtaaaaatgttacgtgaagaaataaatgaaacttggccagtttgttcctctagtagtatatttaattttgacataagtaacttttaaaatttgtcttaaaaatttatacaccagcaatttagacaaagccttaagcaaattttgtattattgttctcacttattattaataatgaagtagaagttacttaattgccagcaaataaatacgtgtcaaaaaagaatctgtattcagaccctgggtcaggaaattactgcccacttgtcaagttcagcccaccatctgtttgaacattatatgaagtttaaattttagtgtccataaataaagtttcagcggaacacaaaaaaaaaaaaaaaaaaaaaaaaaaaaaaaaaaaaaaaaaaaa

SEQ ID NO: 4 - protein, 279 aa, Homo sapiens
MLSPANGEQLHLVNYVEDYLDSIESLPFDLQRNVSLMREIDAKYQEILKELDECYERFSRETDGAQKRRMLHCVQRALIRSQELGDEKIQIVSQMVELVENRTRQVDSHVELFEAQQELGDTAGNSGKAGADRPKGEAAAQADKPNSKRSRRQRNNENRENASSNHDHDDGASGTPKEKKAKTSKKKKRSKAKAEREASPADLPIDPNEPTYCLCNQVSYGEMIGCDNDECPIEWFHFSCVGLNHKPKGKWYCPKCRGENEKTMDKALEKSKKERAYNR

SEQ ID NO: 5 - protein, 210 aa, Homo sapiens
MLHCVQRALIRSQELGDEKIQIVSQMVELVENRTRQVDSHVELFEAQQELGDTAGNSGKAGADRPKGEAAAQADKPNSKRSRRQRNNENRENASSNHDHDDGASGTPKEKKAKTSKKKKRSKAKAEREASPADLPIDPNEPTYCLCNQVSYGEMIGCDNDECPIEWFHFSCVGLNHKPKGKWYCPKCRGENEKTMDKALEKSKKERAYNR

(19) United States
(12) Patent Application Publication
(10) Pub. No.: US 2002/0039735 A1
(43) Pub. Date: Apr. 4, 2002

(54) Monoclonal antibodies to ING1 isoforms

(76) Inventors: Karl T. Riabowol, Calgary (CA); Donna Boland, Calgary (CA)

Correspondence Address: BURNS DOANE SWECKER & MATHIS L L P, POST OFFICE BOX 1404, ALEXANDRIA, VA, 22313-1404, US

(21) Appl. No.: 09/874,347

(22) Filed: Jun. 4, 2001

Related U.S. Application Data
(63) Continuation-in-part of application No. 09/532,868, filed on Mar. 22, 2000.
Application No. 09/532,868 is a division of application No. 09/258,372, filed on Feb. 26, 1999, now Pat. No. 6,238,918.
Application No. 09/258,372 is a continuation of application No. 08/751,230, filed on Nov. 15, 1996, now Pat. No. 6,117,633.
Application No. 08/751,230 is a continuation-in-part of application No. 08/569,721, filed on Dec. 8, 1995, now Pat. No. 6,037,121.
(60) Provisional application No. 60/208,829, filed on Jun. 2, 2000.

(51) Int. Cl. C12Q 1/68; G01N 33/574; C07K 16/30

(52) U.S. Cl. 435/6; 435/7.23; 530/388.8

(57) ABSTRACT

The present invention relates to monoclonal antibodies against the candidate tumor suppressor ING1. A combination of these monoclonal antibodies can detect low levels of endogenous ING1 proteins, and it can also detect at least two different ING1 isoforms, p33ING1b and p47ING1a.

### RELATED APPLICATIONS

This application is a Continuation-In-Part of U.S. patent application Ser. No. 09/532,868, filed Mar. 22, 2000, which was a Divisional application of U.S. patent application Ser. No. 09/258,372, filed Feb. 26, 1999, now U.S. Pat. No. 6,238,918, which was a Continuation of U.S. patent application Ser. No. 08/751,230, filed Nov. 15, 1996, now U.S. Pat. No. 6,117,633, which was a Continuation-In-Part of application Ser. No. 08/569,721, filed Dec. 8, 1995, now U.S. Pat. No. 6,037,121. The content of each of these patents and patent applications is hereby incorporated by reference in its entirety.

This application also claims the benefit of U.S. Provisional Patent Application Ser. No. 60/208,829, filed Jun. 2, 2000, the entire content of which is hereby incorporated by reference in its entirety.

### FIELD OF THE INVENTION

This invention relates to specific monoclonal antibodies which recognize the isoforms of ING1.

### REFERENCES

U.S. Pat. No. 5,965,398.

U.S. Pat. No. 5,986,078.

Boland, D. et al. (2000). A panel of CAb antibodies recognize endogenous and ectopically expressed ING1 protein. Hybridoma 19(2):161-5.

Garkavtsev, I. & Riabowol, K. (1997). Extension of the replicative life span of human diploid fibroblasts by inhibition of the p331NGI candidate tumor suppressor. Mol. Cell. Biol. 17: 2014-2019.

Garkavtsev, I., et al. (1997a). Cellular localization and chromosome mapping of a novel candidate tumor suppressor gene (ING1). Cytogenet. & Cell Genet. 76: 176-178.

Garkavtsev, I., Boland, D., Mai, J., Wilson, H., Veillette, C. and Riabowol, K. (1997b). Specific Monoclonal Antibody Raised Against the p331NGI Tumor Suppressor. Hybridoma 16: 537-540.

Garkavtsev, I., et al. (1998). The candidate tumour suppressor p331NG′ cooperates with p53 in cell growth control. Nature 391: 295-298.

Harlow, E. and Lane, D. (1988). Antibodies: A Laboratory Manual, pages 474-510. Cold Spring Harbor Laboratory, NY.

Laemmli, E. K. (1970). Cleavage of structural proteins during the assembly of the head of the bacteriophage T4. Nature 227: 680-685.

Loewith, R., Meijer, M., Lees-Miller, S., Riabowol, K. And Young, D. (2000). Three yeast proteins related to the human candidate tumor suppressor p33 (ING1) are associated with histone acetyltransferase activities. Mol. Cell Biol. 20, 3807-3816.

Maestro, R. et al (1996). Cancer Res. 56: 1146.

Mitelman, F. et al. (1991). Cytogenet. Cell Genet. 58: 1053.

Motomura, K. et al. (1988). Genomics 2: 180.

Ohmori, M., Tasaka, T., Nagai, M., Fujita, M., Takahara, J., Sonoki, T., Riabowol, K., Koeffler, H. And Tohyama, K. (1999). Decreased expression of p331NGI mRNA in lymphoid malignancies. American J. Hematol. 62: 118-119.

Riabowol, K. et al. (1989). The cdc2 kinase is a nuclear protein that is essential for mitosis in mammalian cells. Cell 57: 393-401.

Zeremski, M., Hill, J., Kwek, S., Grigorian, I., Gurova, K., Garkavtsev, I., Diatchenko, L., Koonin, E. and Gudkov, A. (1999). Structure and Regulation of the Mouse ing I Gene: Three alternative transcripts encode two PHD finger proteins that have opposite effects upon p53 function. J. Biol. Chem. 274, 32172-32181.

All of the cited publications and patents are herein incorporated by reference in their entirety to the same extent as if each individual publication, patent application or patent was specifically and individually indicated to be incorporated by reference in its entirety.

### BACKGROUND OF THE INVENTION

The candidate tumor suppressor gene ING1 (INhibitor of Growth 1) was cloned by using a functional cloning strategy (Garkavtsev et al., 1996). Expression of the ING1 gene is regulated through the cell cycle, decreasing as cells exit G₀, increasing late in G₁ and peaking in the S phase (Garkavtsev and Riabowol, 1997). Its expression is down-regulated from 2 to 10-fold in more than 40% of primary breast tumors (Toyama et al., 1999) and in lymphoid malignancies (Ohmori et al., 1999). Overexpression of a construct containing the ING1 gene inhibited cell growth by reducing the fraction of cells which entered into S-phase. Microinjection of the construct blocked the injected cell from entering into S-phase. These results are all consistent with the role of ING1 as a tumor suppressor gene.

ING1 is located on human chromosome 13q33-34, a region reported to be a site for translocation and deletion in several cancers, including primary gastric cancer, hematologic neoplasms and head and neck squamous cell carcinomas (Motomura et al., 1988; Mitelman et al., 1991; Maestro et al., 1996). To date, a number of ING1 transcripts have been found. Primary and established human cell strains and lines predominantly express three ING1 proteins. p47^{ING1a} and p33^{ING1b} are encoded by two ING1 mRNA isoforms resulted from alternative splicing. p24^{ING1c} is a truncated protein that is believed to be produced by initiation at an internal ATG.

p33^{ING1b}, p47^{ING1a} and p24^{ING1c} share the same C-terminal portion encoded by a common 3′ exon. This shared region contains a PHD domain and a nucleus localization signal. The PHD domain has the consensus sequence Of Cys₄-His-Cys₃. This evolutionarily conserved domain is predicted to chelate two Zn²⁺ ions and is similar to, but distinct from, other zinc binding motifs. PHD domains have been found in many different proteins, including transcription factors and other proteins implicated in chromatin-mediated transcriptional regulation, but the function of the PHD domains has not been determined.

ING1 appears to interact with other cellular factors to regulate the growth properties of cells. For example, the growth-inhibition activity of ING1 is dependent on another tumor suppressor, p53, and vice versa. Neither of these two genes alone causes growth inhibition if the other one is suppressed. Expression of both genes in a mammalian cell results in normal growth regulation and anchorage-dependent growth, and apoptosis is induced in response to irreversible DNA damage and other cellular insult. Inhibition of expression of either gene results in a loss of cellular growth control, anchorage-independent growth, inhibition of apoptosis and resistance to radiation and cytotoxic drugs. Moreover, regulation of gene expression by p53, such as of the p21^{WAF1} gene, also depends on the expression of ING1 (Garkavtsev et al., 1998).

To investigate the roles of the ING1 isoforms and the interaction with p53 or other factors, it is important to have antibodies which can detect the endogenous ING1 proteins, as well as antibodies which recognize the ING1 isoforms. We previously reported a monoclonal antibody, CAb1, which specifically recognizes native and denatured p33ING1b in ELISA and Western blot analyses, respectively (Garkavtsev et al., 1997b). CAb1 is also useful for cellular localization of overexpressed p33ING1 protein by indirect immunofluorescence. However, CAb1 is not sensitive enough to detect the low level endogenous ING1 protein. Moreover, although CAb1 recognizes p33ING1b and a GST-p33ING1 fusion protein, it does not recognize other ING1 isoforms such as p47ING1a and p24ING1c. It is thus desirable to make other monoclonal antibodies against ING1 which can achieve these goals.

### SUMMARY OF THE INVENTION

In the present invention, a panel of monoclonal antibodies, CAb2 to CAb10, were raised against ING1. Combinations of CAbs 1-9 are sensitive enough to detect the endogenous ING1 proteins in normal human diploid fibroblasts and various brain tumor cell lines. A combination of CAbs 1-4 recognize at least two ING1 isoforms. Therefore, this invention provides for useful monoclonal antibodies against ING1, methods to detect endogenous ING1 proteins and methods to detect different ING1 isoforms.

Accordingly, one aspect of the present invention provides a monoclonal antibody which recognizes an epitope of an ING1 protein. In particular, the monoclonal antibody is selected from the group consisting of CAb1, CAb2, CAb3, CAb4, CAb5, CAb6, CAb7, CAb8, CAb9 and CAb10.

The monoclonal antibody is preferably not CAb1. Therefore, the monoclonal antibody is preferably selected from the group consisting of CAb2, CAb3, CAb4, CAb5, CAb6, CAb7, CAb8, CAb9 and Cab10.

Also provides are cells which produce the monoclonal antibody which recognizes an epitope of an ING1 protein. In particular, the monoclonal antibody is selected from the group consisting of CAb1, CAb2, CAb3, CAb4, CAb5, CAb6, CAb7, CAb8, CAb9 and CAb10. The monoclonal antibody is preferably not CAb1. Therefore, the monoclonal antibody is preferably selected from the group consisting of CAb2, CAb3, CAb4, CAb5, CAb6, CAb7, CAb8, CAb9 and CAb10.

Another aspect of the present invention provides a method of detecting an ING1 protein in a cell using monoclonal antibodies, comprising:

(a) selecting a cell;

(b) applying to the cell, extract of the cell, or a form of the cell suitable for the detection of the ING1 protein an anti-ING1 monoclonal antibody or a combination of anti-ING1 monoclonal antibodies in an amount sufficient to detect the ING1 protein in said cell; and

(c) detecting the ING1 protein.

To specifically detect the ING1 protein, the method can further comprise the steps of repeating step (b) wherein the antibody or antibody mix is pre-treated with an ING1 protein and comparing the result obtained with the treated antibody to that obtained with the untreated antibody. If pre-treatment with an ING1 protein results in disappearance of the detected ING1 level, the signal detected with the untreated antibody is completely specific to the ING1 protein used to treat the antibody. Similarly, if the pre-treatment results in a reduction in the signal, then the difference between levels detected by the untreated and pre-treated antibody is specific to the ING1 protein used to treat the antibody.

The anti-ING1 monoclonal antibody used in this method is preferably an antibody selected from the group consisting of CAb2, CAb3, CAb4, CAb5, CAb6, CAb7, CAb8, CAb9 and CAb10. More preferably, the antibody is a combination of two or more antibodies selected from the group consisting of CAb1, CAb2, CAb3, CAb4, CAb5, CAb6, CAb7, CAb8, CAb9 and CAb10. Most preferably, the combination is a mixture of CAb1 to Cab10.

This method can be used to detect endogenous ING1 or ectopically expressed ING1 proteins.

Yet another aspect of the present invention provides for a method of detecting at least two different isoforms of ING1 with monoclonal antibodies, comprising using an anti-ING1 monoclonal antibody or a combination of anti-ING1 monoclonal antibodies under conditions which result in the detection of at least two isoforms of ING1. The combination if preferably two or more antibodies selected from the group consisting of CAb1, CAb2, CAb3, CAb4, CAb5, CAb6, CAb7, CAb8, CAb9 and CAb10. More preferably, the combination is a mixture of CAb1, CAb2, CAb3, and CAb4. The ING1 isoforms may be ectopically or endogenously expressed in a cell.

Also provided are kits comprising anti-ING1 monoclonal antibodies and secondary antibodies which recognize the anti-ING1 monoclonal antibodies. These kits are useful in detecting ING1 proteins in immunofluorescence assays, Western blot assays, ELISAs or any antibody-mediated detection method established in the art. Accordingly, the kit may further comprise additional reagents or devices for each individual assay. For example, a kit for Western blot assay my further comprise reagents for making the SDS-PAGE gel as well as blot membranes and buffers for the blotting.

Another aspect of the present invention provides a method of diagnosing a disease or medical condition in an animal associated with aberrant levels of an ING1 protein, comprising determining levels of the ING1 protein with a monoclonal antibody or a combination of monoclonal antibodies which is capable of recognizing the ING1 protein. The disease is preferably a tumor. The levels of ING1 proteins can be determined using a sample obtained from the animal, such as a biopsy sample of the tumor. The animal is preferably a human.

### DETAILED DESCRIPTION OF THE INVENTION

This invention relates to monoclonal antibodies against the ING1 candidate tumor suppressor.

As used herein, “ING1” refers to an ING1 gene. An ING1 gene is a gene which has substantial homology with the human ING1 gene and which encodes a protein with ING1 biological activities.

A gene has substantial homology with the human ING1 gene if the gene encodes a protein which has at least about 60% amino acid sequence identity with p47ING1a (SEQ ID NO:2), p33ING1b (SEQ ID NO:4) or p24ING1c (SEQ ID NO:5). The sequence identity is more preferably at least about 70%, still more preferably at least about 80%, and most preferably at least about 90%. Alternatively, a gene having substantial homology with the human ING1 gene is the gene hybridizes under stringent conditions to the human ING1 gene having the sequence of SEQ ID NO: 1 (p47ING1a gene) or SEQ ID NO:3 (p33ING1b gene). “Stringent conditions”, in turn, refer to hybridization conditions at a minimal stringency equivalent to 0.1 times SSC, 0.1% SDS and 55° C. The stringency is preferably 0.1 times SSC, 0.1% SDS and 60° C., and more preferably 0.1 times SSC, 0.1% SDS and 65° C.

“ING1 biological activities” means at least one of the following: (1) immunological cross-reactivity with a polyclonal antiserum raised against the native p47ING1a, p33ING1b or p24ING1c protein; or (2) an activity to inhibit cell proliferation or tumor formation, which activity is possessed (but maybe at a different level) by the native p47ING1a, p33ING1b or p24ING1c protein.

Cross-reactivity can be determined by any method established in the art, such as diffusion assays or ELISA. Assays for cell proliferation inhibition or tumor formation inhibition are also well-known to skilled artisans (for example, see U.S. Pat. Nos. 5,965,398 and 5,986,078). Examples of these activities include the activity to inhibit cell growth as measured by the decrease of the S-phase fraction in a population of cells, the activity to inhibit focus formation in soft agar, the activity to inhibit cell proliferation as determined by the number increase of cells in a population over time, the activity to reduce tumor size or mass, and the activity to inhibit tumor cell metastasis.

While ING1 was originally cloned from a human cDNA library, mouse and yeast ING1 homologs have also been identified by nucleic acid sequence similarity (Zeremski et al., 1999; Loewith et al., 2000). It is contemplated that ING1 also encompasses the mouse and yeast ING1 genes.

As used herein, “ING1 protein” or “ING1 gene product” refer to a protein encoded by an ING1 gene. Examples of ING1 proteins include, but are not limited to, p47ING1a, p33ING1b and p24ING1c.

A “disease or medical condition associated with aberrant levels or an ING1 protein” is a disease or medical condition which is associated with abnormally high or abnormally low levels of a ING1 protein. For example, as previously disclosed, ING1 is down-regulated from 2 to 10-fold in more than 40% of primary breast tumors (Toyama et al., 1999) and in lymphoid malignancies (Ohmori et al., 1999).

A “sample” is a specimen obtained from an animal, such as blood, urine, a piece of tissue, hair, saliva, and the like.

To raise monoclonal antibodies against the ING1 proteins, a GST-ING1 fusion protein was used to immunize BALB/c mice, and spleen cells from the immunized mice were fused with Sp2/mIL6 myeloma cells to make hybridomas. This GST-ING1 fusion protein was expressed from a plasmid containing nucleotides 161 to 1143 of the human ING1 gene. Synthesis of this plasmid and preparation of this GST-ING1 fusion protein were performed as previously described (Garkavtsev et al., 1997b). Hybridomas were produced as described in Materials and Methods.

In three previous attempts, it had proven difficult to isolate stable colonies of hybridomas expressing antibodies directed against the ING1 proteins. Many positive colonies were detected initially, but they were unstable and rapidly lost their titer. We noted during the course of these experiments that stable hybridoma lines came from those mice which did not have the highest titer as determined by ELISA. Moreover, we initially selected the mice whose sera had high reactivity with the ING1 portion of the GST-ING 1 fusion protein and reasonably low cross-reactivity with GST. However, hybridoma fusion results indicated that stable hybridoma clones were most frequently derived from the mice with high reactivity to ING1 and medium to high GST cross-reactivity.

Nine hybridomas were obtained which reacted specifically with ING1 but not the GST moiety. The antibodies produced by these nine hybridomas are designated CAb2 to CAb10, respectively. High levels of specificity of these antibodies are demonstrated in Example 1. Thus, each of CAbs 1-4 could detect the GST-ING1 fusion protein at concentrations as low as 5 ng/ml, while none of them recognized GST, even at 500 ng/ml.

To determine if these antibodies can detect endogenous ING1, a mixture of CAbs 1-9 was used to stain Hs68 fibroblasts or brain cancer cell lines. As used herein, “endogenous ING1” refers to the ING1 proteins expressed in a cell which has not received any exogenously added ING1 gene, RNA or protein. As shown in Example 2, the antibody mix was able to detect the endogenous ING1 proteins in both Hs68 fibroblasts and the brain cancer cells. The antibody mix stained both the nucleus and cytoplasm, but when the mix was pre-treated with the GST-ING1 fusion protein, the nuclear signal disappeared, indicating that the nuclear staining was specific for ING1. This result is consistent with the previous report that the ING1 protein is localized in the nucleus.

These antibodies are also useful in Western blot analyses. A mixture of CAb1-4 was used to stain Western blots of various cell extracts (Example 3) and recognized p33ING1b in cells transfected with a vector containing the ING1 sequence. The antibody mix also recognize the same band in a variety of brain tumor cell lines and a weak band corresponding to p47ING1a. Therefore, these antibodies recognize at least two isoforms of ING1 and will be useful in determining the functions and mechanisms of the isoforms. For example, cellular extracts can be immunoprecipitated with an antibody against another protein which is suspected of interacting with the ING1 proteins, and the immunoprecipitates can be blotted with a mixture of the CAb1-4 antibodies to determine which isoforms are involved.

The activity of each individual antibody in Western blots is shown in Example 4. Every antibody recognized p33ING1b. The relative activity of the antibodies in ELISA, immunofluorescence and Western blot is summarized in Table 1.

It is contemplated that more monoclonal antibodies may be produced using different portions of ING 1 or different ING1 isoforms as antigens. The resulting hybridomas can be tested as described herein in order to obtain antibodies which can distinguish the ING1 isoforms, or to obtain antibodies with higher sensitivity. Also contemplated is the identification of antibodies which recognize different portions of the ING1 proteins. Such antibodies can be used in structure-function analysis of the ING1 proteins.

Recombinant derivative antibodies can be made using the hybridomas described herein according to well-known genetic engineering methods. A review for these methods can be found in Winter et al., Nature 349: 293, 1991. For example, the DNA fragment coding for the variable regions of the monoclonal antibodies can be obtained by polymerase chain reactions (PCR). The PCR primers can be oligonucleotides which are complementary to the constant regions of the heavy chain or light chain, and the PCR template can be the total cDNA or genomic DNA prepared from the hybridomas. Alternatively, a cDNA library can be prepared from the hybridomas and screened with probes which correspond to the constant regions of immunoglobulin heavy chain or light chain to obtain clones of the heavy chain or light chain produced by the particular hybridoma.

Subsequently, the DNA fragment for the variable regions can be inserted into an expression vector and joined in frame with the cDNA sequences of a selected constant region. The constant region can be the human constant sequences to make humanized antibodies, the goat constant sequences to make goat antibodies, the IgE constant sequences to make IgE which recognizes the ING1 proteins, and the like. Thus, antibodies with the same antigen recognition ability but different constant regions can be produced. Such antibodies will be useful, for instance, in indirect immunofluorescence experiments where the choice of secondary antibodies is limited. For example, if an anti-p53 antibody and an anti-ING 1 antibody will be used in the same experiment but both are mouse antibodies of the same isotype, it will not be easy to distinguish the two signals. An anti-ING1 antibody of another kind of constant region will solve this problem.

The monoclonal antibodies can also be used to produce anti-idiotypic antibodies. To make anti-idiotypic antibodies, hybridoma cells for each monoclonal antibody can be used as antigens to immunize an animal for antibody productions. Alternatively, polypeptides corresponding to the variable region of the monoclonal antibodies can be expressed in a suitable host system using DNA fragments prepared as described above, and used as antigens in immunizations.

The following examples are offered to illustrate this invention and are not to be construed in any way as limiting the scope of the present invention.

### EXAMPLES

In the examples below, the following abbreviations have the following meanings. Abbreviations not defined have their generally accepted meanings.

° C.=degree Celsius

hr=hour

min=minute

μM=micromolar

mM=millimolar

M=molar

ml=milliliter

μl=microliter

mg=milligram

μg=microgram

PAGE=polyacrylamide gel electrophoresis

rpm=revolutions per minute

FBS=fetal bovine serum

DTT=dithiothrietol

SDS=sodium dodecyl sulfate

PBS=phosphate buffered saline

DMEM=Dulbecco's modified Eagle's medium

α-MEM=α-modified Eagle's medium

β-ME=β-mercaptoethanol

DMSO=dimethylsulfoxide

ELISA=enzyme-linked immunosorbent assay

CMV=cytomegalovirus

GST=glutathione-S-transferase

IPTG=isopropyl beta-D-thiogalactoside

### MATERIALS AND METHODS

HYBRIDOMA

Four female BALB/c mice (5- to 6-weeks old) were boosted with 10 μg each of recombinant GST-ING1 protein prepared as previously described (Garkavtsev et al., 1997b). The protein in the first injection was emulsified with Freund's complete adjuvant and with Freund's incomplete adjuvant for subsequent injections, with injections being done every three weeks. After a series of three injections the sera were tested by ELISA and 2 mice with the best titers were selected for fusion.

Four days before the fusion, the mice were boosted by tail vein injections with 5 μg of GST-ING1. On the day of the fusion, spleens were removed from the mice, and spleen cells were isolated and fused with Sp2/mIL6 myeloma cells using polyethylene glycol 1500 as the fusing agent. The cells were resuspended in media containing hypoxanthine/aminopterin/thymidine (HAT) to select for hybridoma growth and were plated into 96-well tissue culture plates. After 10 to 14 days the colonies were tested by ELISA and positive colonies were subcloned twice and grown up to larger volumes for further testing. The cells were adapted into SFM, a serum free medium (Gibco BRL) and allowed to produce concentrated supernatant for use in western blotting and indirect immunofluorescence assays following microinjection of ING1 expression construct into various cell types. All the cell lines that were obtained grew well in SFM.

ELISA

The ELISA method was used to test emergent colonies for titer. 100 μl aliquots of cell supernatant were added to individual wells of 96 well ELISA plates previously coated with GST-ING1 and incubated for 1 hour at 37 ° C. Plates were then washed with PBS/0.1% Tween 20, pH 7.4 (wash buffer). Goat anti-mouse IgG-biotin was diluted to 1/1000 with 1% BSA/PBS/0.1% Tween 20, pH7.4 (diluting buffer) and was added to the wells, incubated for 1 hour at 37 ° C. and washed 5 times with wash buffer. Strepavidin-alkaline phosphatase was diluted to 1/1000 with diluting buffer, incubated and washed as per the previous step. Paranitrophenylphosphate in substrate buffer was used as the developing agent. The plate was read at OD 405 and any positives were re-tested two days later against both GST-ING1 and GST to determine cross-reactivity. To determine the subisotypes of the various hybridoma lines a MouseHybridoma-Subtyping Kit (Boehringer Mannheim) was used according to the manufacturer's directions.

MICROINJECTION, FIXATION AND IMMUNOFLUORESCENCE

For microinjection studies, Hs68 cells were plated on acid-washed 1 cm glass coverslips and allowed to grow for 48 hr prior to microinjection. Sense or antisense orientations of the a or b forms of ING1 cDNA cloned downstream of the CMV promoter in the pC1 vector (Promega), or the vector alone, were diluted to 0.1 mg/ml in sterile PBS as described (Garkavtsev et al., 1996), and solutions were centrifuged for 5 minutes at 14,000×g to remove particulate material. Solutions containing expression or control plasmids were injected into the nuclei of cells using needle microcapillaries and cells were subsequently incubated in complete medium for 24 hr.

After this time period, cells were fixed by immersion in 3.7% formaldehyde diluted in PBS (pH 7.0) for 10 min at room temperature (RT) and permeabilized with 0.5% Triton X-100 in PBS for an additional 10 min at RT. Coverslips were stored at 4° C. overnight (12-24 hr) in sterile PBS prior to staining. In the case of controls and cells injected with antisense constructs, no staining above background levels was observed. For subsequent studies, only uninjected cells and cells expressing ING1 were recorded photographically following staining for ING1 protein.

Hybridoma supernatants derived from 10 individually isolated monoclonal antibodies including CAb1 (Garkavtsev et al., 1997b) were used undiluted as primary antibodies and staining was compared to a negative control consisting of mouse myeloma supernatant. A polyclonal rabbit antiserum diluted 1:200 that has been described previously (Garkavtsev et al., 1997a) was used as a positive control for indirect immunofluorescence. Coverslips were incubated with 50 μl of primary antibody solution for 30 minutes at 37° C., washed 3 times for 10 min in PBS, and incubated with biotinylated goat anti-rabbit IgG or biotinylated goat anti-mouse IgG that were diluted 1:200 with PBS containing 0.1% bovine serum albumin (BSA). Coverslips were again washed 3 times for 10 minutes in PBS and incubated with a 1:150 dilution of Strepavidinconjugated Texas Red (in 0.1% BSA/PBS) for 30 min before washing and mounting of coverslips in Gelvatol containing n-propyl gallate as an antiquenching agent as described (Riabowol et al., 1989). Cells were visualized on a Zeiss Axiophot microscope and images were recorded using Kodak Tri X-100 film.

WESTERN BLOTTING

Individual 10 cm petri dishes of cells were harvested in 0.5 ml Laemmli sample buffer (Laemmli, 1970) and equivalent protein loading was estimated by Coomassie Brilliant Blue staining of protein samples resolved using sodium dodecylsulfate polyacrylamide gel electrophoresis (SDS-PAGE). For western blots, samples were resolved by SDS-PAGE and transferred to nitrocellulose membranes as described (Harlow and Lane, 1988). The membranes were blocked overnight in phosphate buffered saline (PBS) containing 0.1% Tween 20 (PBS-Tween) and 5% powdered skim milk. All subsequent incubations were performed for 30 minutes at room temperature and washes were performed three times each for 5 minutes in PBS-Tween. Membranes were incubated in primary antibodies (polyclonal a-GST-ING1, 1:1000 to 1:5000; monoclonal a-ING1 tissue culture supernatants diluted 1:1 with PBS-Tween-5% non-fat milk), washed, incubated with secondary antibodies (biotinylated goat a-rabbit or mouse, 1:1000; Amersham), washed and incubated with tertiary reagent (strepavidin-conjugated horseradish peroxidase, 1:1000; Amersham). After a final wash, membranes were treated with chemiluminescent reagent (ECL reagent, Amersham) and exposed for 3 minutes to 1 hour to X-ray film (Kodak OMAT).

CELL TRANSFECTION

For transfection of primary human fibroblasts (Hs68) or the brain tumor line SNB19, cells were grown in 15 cm petri dishes (Corning) in Dulbecco's Modified Eagles medium (DMEM) (Gibco) supplemented with 10% fetal bovine serum (FBS) (Gibco) to approximately 85 to 90% confluence. Each dish contained cells for one transfection. Cells were trypsinized, pelleted and resuspended in 400 μl DMEM without FBS. 10 μg of either pCl-ING1b or pCl vector (Promega) plus 10 μg of sheared salmon sperm DNA (Amersham Pharmacia) were made up to 100 μl in DMEM without FBS and this was added to each cell suspension. Each 500 μl aliquot of cell suspension plus DNA mix was transferred to a sterile 4 mm gap electroporation cuvette (BTX). Electroporator (BioRad) settings were 250 V, 960 μFd and capacitance, EXT (extender). After electroporation, cells were transferred to 10 cm dishes with 10 ml DMEM containing 10% FBS for 24 hours and were then re-fed. After a further 24 hours the cells were harvested for protein analysis by scraping in Laemmli sample buffer and boiling for 1 min.

### Example 1

### Detection of ING1 in ELISA by CAb1-4

The ELISA plate was coated with GST-ING1 or GST in alternate rows. The concentration of GST-ING1 or GST ranged from 5, 10, 5, 100 to 500 ng/ml in each well. CAb1-4 was used to detect GST-ING1 or GST as described in Materials and Methods, and the conditioned medium from Sp2/mIL6 myelomas served as a negative control.

The results indicate that CAb1-4 were all capable of detecting the lowest level of GST-ING1 while none of them cross-reacted with GST, even when the concentration of GST was as high as 500 ng/ml. The negative control yielded no signal as expected. Therefore, these monoclonal antibodies are highly specific for the ING1 protein.

CAb5-10 were also tested and each staining intensity was measured with a Biomek 1000 ELISA reader. The resulting readings were used to generate the ELISA ranking shown in Table 1. Each of the monoclonal antibodies displayed a titer at least comparable to that of a mouse polyclonal antiserum, the titer of which is arbitrarily set to be 1.00.

### Example 2

### Indirect Immunofluorescence of Endogenous and Ectopically Expressed ING1

To test if the CAbs can detect ectopically expressed ING1 proteins in indirect immunofluorescence assays, individual cells were microinjected with an ING1 expression construct containing the CMV promoter. The cells were then fixed and stained with nuclear staining as well as different ING1 antibodies. A rabbit polyclonal antibody against ING1 proteins yielded strong staining signals, indicating that the injected gene was expressed at a high level. The mixture of CAb1-4 or each monoclonal antibody alone was also capable of generating positive staining. The results are summarized in Table 1 where the relative strength of nuclear staining is listed. The results indicate that these antibodies or antibody mixtures can detect the ING1 proteins in individual cells.

To determine if these antibodies can detect endogenous ING1 proteins, a mixture of CAb1-9 was used to stain Hs68 fibroblasts which are known to express ING1 proteins. Clear nuclear and cytoplasmic signals were observed. However, when the antibody mix was blocked with the GST-ING1 fusion protein prior to staining, the nuclear signal, but not the cytoplasmic signal, was markedly reduced. This is consistent with the previous observation that ING1 products are localized to the nucleus. Similarly, brain cancer cell lines known to express much higher levels of ING1 show clear nuclear signals when stained with the monoclonal mix. Therefore, these antibodies are sensitive enough to detect endogenous ING1 proteins.

### Example 3

### Western Blot Assays of Endogenous and Ectopically Expressed ING1 Proteins with CAb Mixtures

An equal mixture of CAb1-4 was used to detect ING1 in extracts of transfected fibroblast cells, bacterial cells harboring an expression construct for ING1, and a number of human brain tumor cell lines in Western blot assays.

Hs68 fibroblasts transfected with pC1-ING1b produced a clear band migrating at a position expected of p33ING1b. In contrast, Hs68 fibroblasts transfected with pC1 vector alone showed essentially no signal at this position. Similarly, bacterial lysates from bacteria harboring an IPTG-inducible GST-ING1 expression construct yielded a strong 58 kD band which corresponds to the GST-ING1 fusion protein band only in the presence of IPTG. In the absence of IPTG, no signal could be observed. Clearly, the monoclonal antibodies can be used in Western blot assays to detect ING1 proteins.

Whole cell lysates of various human brain tumor lines were also used to test the ING1 antibodies. These tumor lines include SF188, U251, U373, TE671, SNB19, HBS683 and U343. The CAb mix recognized a 33kD band and a weak band at approximately 47 kD, which corresponds to p47ING1a. Therefore, the CAb mix recognizes at least two isoforms of ING1.

### Example 4

### Western Blot Assays with Individual CAb1-9

The ability of each individual antibody to recognize ING1 in Western blot assays was assessed using the conditioned medium from the myeloma line Sp2/mIL6 as a negative control. The results show that each of CAb1-9 binds to p33ING1b to a different degree. The relative strengths of the antibodies were estimated visually and included in Table 1, where the strength of the mouse polyclonal antiserum was arbitrarily assigned the value of 1.

TABLE 1
Characterization of the CAb line of monoclonal antibodies by sub-isotyping, comparative
titer, intensity and degree of nuclear localization and band mobility via western blotting.

| | | | | | | | | | | | MOUSE | RABBIT |
|---|---|---|---|---|---|---|---|---|---|---|---|---|
| NAME | CAb 1 | CAb 2 | CAb 3 | CAb 4 | Cab 5 | CAb 6 | CAb 7 | CAb 8 | CAb 9 | CAb 10 | POLY | POLY |
| SUB-ISOTYPE | IgG2a | IgG1 | IgG1 | IgG1 | IgG2a | IgG1 | IgG1 | IgG1 | IgG1 | IgM | — | — |
| ELISA RANKING | 1.00 | 1.13 | 1.78 | 1.74 | 0.85 | 1.92 | 2.30 | 2.36 | 1.14 | 1.10 | 1.00 | — |
| NUCLEAR | + + + + | + + + | + + + + | + + | + + + | + + + + | + + + | + + + | + + + + | NOT | + + + | + + + + |
| LOCALIZATION | | | | | | | | | | TESTED | | |
| (IMMUNO- | | | | | | | | | | | | |
| FLUORESCENCE) | | | | | | | | | | | | |
| WESTERN BLOT | + + + + | + + + + | + + + + | + + + + | + | + + | + + + + | + + | + + + | NOT | + | NOT |
| 33 kD BAND | | | | | | | | | | TESTED | | TESTED |
| STRENGTH | | | | | | | | | | | | |

We claim:

1. A monoclonal antibody which specifically recognizes an epitope of an ING1 protein.

2. The antibody of claim 1 wherein the antibody is selected from the group consisting of CAb1, CAb2, CAb3, CAb4, CAb5, CAb6, CAb7, CAb8, CAb9 and CAb10.

3. The antibody of claim 1 wherein the antibody is not CAb1.

4. The antibody of claim 3 wherein the antibody is selected from the group consisting of CAb2, CAb3, CAb4, CAb5, CAb6, CAb7, CAb8, CAb9 and Cab10.

5. A cell producing the antibody of claim 1.

6. A cell producing the antibody of claim 2.

7. A cell producing the antibody of claim 3.

8. A cell producing the antibody of claim 4.

9. A method of detecting an ING1 protein in a cell using anti-ING1 monoclonal antibodies, comprising:
(a) selecting a cell;
(b) applying to the cell, extract of the cell, or a form of the cell suitable for the detection of the ING1 protein an anti-ING1 monoclonal antibody or a combination of anti-ING1 monoclonal antibodies in an amount sufficient to detect the ING1 in said cell; and
(c) detecting the ING1 protein.

10. The method of claim 9 further comprising the steps of:
(d) repeating step (b) wherein the monoclonal antibody or combination of monoclonal antibodies is pre-treated with an ING1 protein prior to application to the cell, extract of the cell, or a form of the cell suitable for the detection of the ING1 protein;
(e) observing the result of step (d); and
(f) comparing the detection level of step (c) to that of step (e).

11. The method of claim 9 wherein the anti-ING1 monoclonal antibody is selected from the group consisting of CAb2, CAb3, CAb4, CAb5, CAb6, CAb7, CAb8, CAb9 and CAb10.

12. The method of claim 9 wherein the combination of anti-ING1 monoclonal antibodies is a combination of two or more antibodies selected from the group consisting of CAb1, CAb2, CAb3, CAb4, CAb5, CAb6, CAb7, CAb8, CAb9 and CAb10.

13. The method of claim 12 wherein the combination of anti-ING1 monoclonal antibodies is a mixture of CAb1, CAb2, CAb3, CAb4, CAb5, CAb6, CAb7, CAb8 and CAb9.

14. The method of claim 9 wherein the ING1 protein is ectopically expressed in the cell.

15. The method of claim 9 wherein the ING1 protein is endogenously expressed in the cell.

16. A method of detecting at least two different isoforms of ING1 with monoclonal antibodies, comprising using an anti-ING1 monoclonal antibody or a combination of anti-ING1 monoclonal antibodies under conditions which result in the detection of at least two isoforms of ING1.

17. The method of claim 16 wherein the combination of anti-ING1 monoclonal antibodies is a combination of two or more antibodies selected from the group consisting of CAb1, CAb2, CAb3, CAb4, CAb5, CAb6, CAb7, CAb8, CAb9 and Cab10.

18. The method of claim 17 wherein the combination of anti-ING1 monoclonal antibodies is a combination of CAb1, CAb2, CAb3 and Cab4.

19. The method of claim 16 wherein the isoforms of ING1 are ectopically expressed in a cell.

20. The method of claim 16 wherein the isoforms of ING1 are endogenously expressed in a cell.

21. A kit comprising:
(a) a monoclonal antibody against an ING1 protein or a combination of monoclonal antibodies against an ING1 protein; and
(b) a secondary antibody which recognizes the monoclonal antibody or antibodies.

22. The kit of claim 21 which is useful in detecting the ING1 protein in an immunofluorescence assay.

23. The kit of claim 21 which is useful in detecting the ING 1 protein in a Western blot assay.

24. A method of diagnosing a disease or medical condition in an animal associated with aberrant levels of an ING1 protein, comprising determining levels of the ING1 protein with a monoclonal antibody or a combination of monoclonal antibodies which is capable of recognizing the ING1 protein.

25. The method of claim 24 wherein the disease is a tumor.

26. The method of claim 24 wherein the levels of the ING1 protein is determined using a sample obtained from the animal.

27. The method of claim 24 wherein the animal is a human.
